(19) United States
(12) Patent Application Publication
(10) Pub. No.: US 2011/0182821 A1
(43) Pub. Date: Jul. 28, 2011

(54) CLUSTERED MAGNETIC PARTICLES AS TRACERS FOR MAGNETIC PARTICLE IMAGING

(75) Inventors: Holger Gruell, Eindhoven (NL); Hans Marc Bert Boeve, Hechtel-Eksel (BE); Denis Markov, Veldhoven (NL)

(73) Assignee: KONINKLIJKE PHILIPS ELECTRONICS N.V., EINDHOVEN (NL)

(21) Appl. No.: 12/674,444

(22) PCT Filed: Aug. 28, 2008
(86) PCT No.: PCT/IB2008/053461

§ 371 Date: Feb. 22, 2010

(30) Foreign Application Priority Data
Aug. 31, 2007 (EP) 07115427.2

(51) Int. Cl. A61K 49/06; A61K 49/18; B29C 31/10

(52) U.S. Cl. 424/9.3; 264/108

(57) ABSTRACT

The invention provides a magnetic tracer material for use in magnetic particle imaging and a manufacturing method thereof. The magnetic tracer material comprises clusters of a plurality of magnetic particles that are clustered in a controlled way to form individual entities, for example, stabilized oil droplets, solid emulsion particles, liposomes, polymersomes or vesicles, or naturally occurring biological entities such as cells or viruses.

### FIELD OF THE INVENTION

The invention relates to magnetic particle compositions for use as magnetic tracers in Magnetic Particle Imaging (MPI) applications.

### BACKGROUND OF THE INVENTION

Magnetic Particle Imaging (MPI) allows direct 3D imaging of magnetic materials. Spatial images are produced by measuring the magnetic fields generated by magnetic particles introduced in an examination area. As described, for example, in US 2003/0085703 A1, a spatially inhomogeneous magnetic field is generated in the examination area, the magnetic field including a first zone in which the magnetization of the particles is in a non-saturated state. In the remaining portion of the examination zone, the magnetic field is strong enough to keep the particles in a state of saturation. Shifting the first zone within the examination area produces a change in the magnetization which can be externally detected and contains information concerning the spatial distribution of the magnetic particles in the examination zone. Conveniently, a time-varying sinusoidal magnetic field is applied which induces higher harmonics which are detected and evaluated.

The signal intensity of magnetic tracers in MPI thus correlates with the intensity of the higher harmonics picked up upon re-magnetizing the magnetic tracers in a high frequency RF field. Therefore, the performance of magnetic tracers in MPI is dependant on their magnetic properties in a RF field, which again is linked to the material and magnetic properties of singular particles but also on possible interactions between a number of particles.

A method of determining changes in parameters such as temperature, pH or clustering using MPI techniques is described in WO 2004/091397 A2. The method makes use of the effect that magnetic particles change their properties when they are close together and thus under the influence of each other's magnetic field. The response of the individual particles to an external magnetic field is changed due to the coupling with the magnetic fields of the neighboring particles. WO 2004/091397 A2 further describes a variety of tracers suitable for use in the method described therein, e.g. functionalized particles, particle clusters or complexes, particles in gels or other spatially delimited media. However, there remains the need for magnetic tracers having improved MPI performance.

### SUMMARY OF THE INVENTION

It would be desirable to provide a new and improved tracer material for use in MPI having well-defined magnetic properties, and a method for producing such a material.

The present invention provides a magnetic tracer material for use in magnetic particle imaging and a method for manufacturing the magnetic tracer material. The magnetic tracer material comprises clusters of a plurality of magnetic particles that are clustered in a controlled way to form individual entities, for example stabilized oil droplets, solid emulsion particles, liposomes, polymersomes or vesicles. Preferably, the magnetic particles are of a well controlled composition, e.g. Fe₂O₃, Fe₃O₄, or, generally, FeₓO_{y}, or doped materials, e.g. Co, Ni, Zn, Mn, . . . :FeₓO_{y} materials, or other magnetic materials such as Fe, Co, Ni, or any other magnetic material such as ferrites, e.g. MnZn. Furthermore, the magnetic particles can be protected from the environment by a non-magnetic shell, called core-shell particles. Examples are magnetic cores formed by above mentioned magnetic materials encapsuled by a non-magnetic shell with a thickness in the range of nanometers of for example, gold, silicon dioxide, non-magnetic iron-oxides or organic coatings.

The magnetic particles have a suitable and shape. In case particles that contribute to the MPI signal with and without interaction with others are clustered, the diameter of the particles should preferably be in the range of 20-50 nm in order to give sufficient MPI signal. The advantage is that there is a large concentration on one spot, leading upon image acquisition to a so-called hot-spot. The interaction between the particles may be constructive or destructive for the MPI signal.

Alternatively, also smaller particles or grains, with diameters in the range of e.g. 5-10 nm, may be deliberately clustered. Such smaller particles can be manufactured with a very monodisperse distribution. In case such smaller particles are clustered, the interaction between particles is a necessity to yield a good MPI signal, and therefore can be prone to changes in temperature, pH, etc. That effect can be exploited to design smart probes that change signal depending on their biological environment, i.e. increased temperature in inflamed tissue, or slightly acidic pH in hypoxic tumors or within the cytosol. The clustered smaller particles form a single entity with a size range between tens to hundreds of nm.

The clustered smaller particles may further form a first sub-entity that is equivalent to a 20-50 nm single magnetic particle to yield optimal MPI properties. Examples include multi-grain particles as well as chemically stabilized ordered structures in a protected environment, i.e. an emulsion. Such sub-entities can then be clustered into individual entities in which the interaction between sub-entities, or magnetic particles within different sub-entities, may be constructive or destructive for the MPI signal.

The individual entities may have a size of between 10 and 1000 nm, preferably between 100 to 200 nm in diameter, so that one of the individual entities may include up to about 1000 or more magnetic particles.

The individual entities are thus formed from controlled clustering of a plurality of well defined smaller particles. To this end, a number of smaller magnetic particles are collected in a spatially dispersed medium, e.g. oil, vesicle etc. The clustering can then take place in a controlled way, e.g. using control of the temperature or the magnetic field, so that the dispersity of the magnetic properties of the entities, such as the size and/or anisotropy, can be controlled. Moreover, the entities which may be present in solution, may optionally be further stabilized.

Arranging, in an individual entity, a controlled number of magnetic particles and/or magnetic particles in a controlled arrangement will lead to new and improved magnetic properties of the whole entity in MPI compared to a single magnetic particle due to the interaction of the magnetic particles in the individual entity. An improved monodispersity in the magnetic properties of the individual entities may provide benefits in numerical quantification and validation of MPI as an imaging technique for molecular medicine.

Another aspect of the invention is the possibility that unclustered particles differ in their signal with respect to controlled clustered particles. Thus, bio-induced clustering of particles may allow to image biological processes such as cell uptake of particles where cell uptake leads to a clustering within the cell and thus to a change in signal. Medical application of this effect lies in cell tracking or in imaging of macrophage uptake of magnetic particles. The same effect can be exploited by using for example red blood cells containing magnetic particles for imaging. Other biological entities providing the option to carry clustered particles are possible as well, examples are viruses, nanocapsules etc.

These and other aspects of the invention will be apparent from and elucidated with reference to the embodiments described hereinafter.

### BRIEF DESCRIPTION OF THE DRAWINGS

FIG. 1 shows a transmission electron microscopy (TEM) picture of hydrophobic coated magnetic particles.

FIG. 2 shows the result of a dynamic light scattering (DLS) measurement of magnetic particles suspended in toluene.

FIG. 3 shows the result of an X-ray diffraction measurement of magnetic particles.

### DETAILED DESCRIPTION OF EMBODIMENTS

A magnetic tracer material comprising clusters of magnetic particles may comprise emulsion based entities. Emulsion based entities may be synthesized using the following process steps:

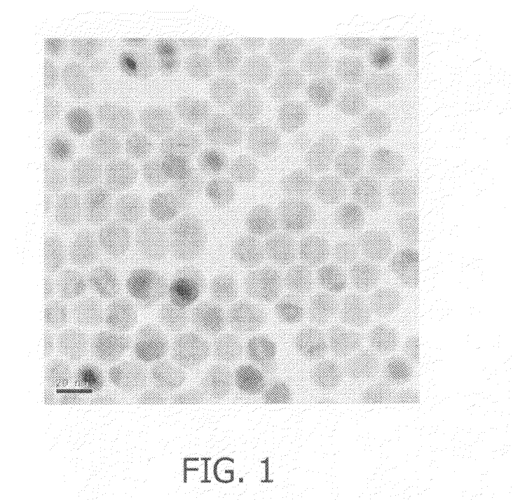

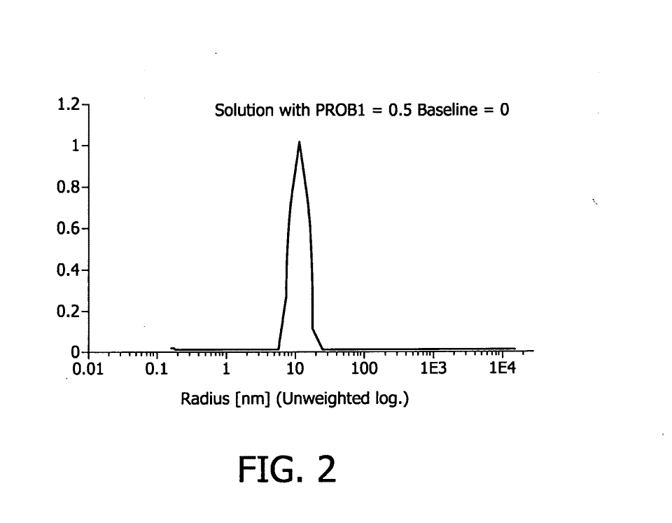

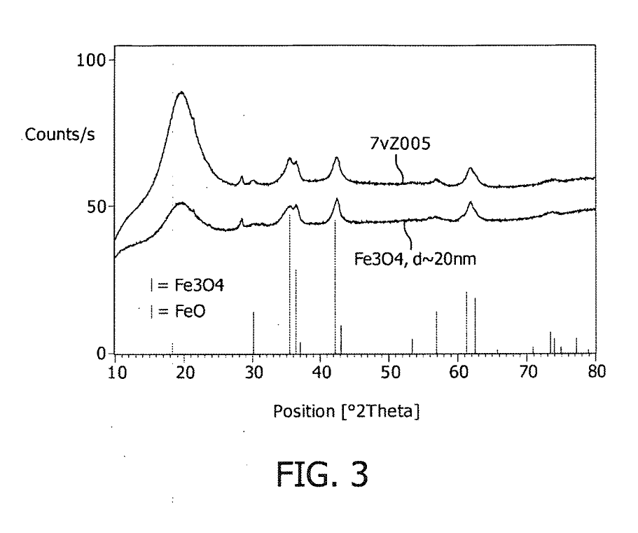

In a first step, magnetic particles of a well controlled composition, e.g. Fe₂O₃, Fe₃O₄, or, generally, FeₓO_{y}, or doped materials, e.g. Co, Ni, Zn, Mn, . . . :FeₓO_{y} materials, other magnetic materials such as Fe, Co, Ni, or any other magnetic material of any suitable and well-controlled shape and size are synthesized, preferably in a way that the particles get an hydrophobic surface coating. Examples of the synthesis of such materials are known in the art. In FIG. 1, a TEM picture of hydrophobic coated iron oxide particles is shown. FIG. 2 shows the result of a DLS measurement, whereas in FIG. 3 the result of an X-ray diffraction measurement of iron oxide particles having a size of 20 nm and a hydrophic surface coating of oleic acid is shown. These particles are stable and well dispersed in hydrophobic organic solvents such as toluene, heptane or CH₂Cl₂.

The particles are also stable in hydrophobic oils such as safflower oil or poppy seed oil or (partly) fluorinated oils or at elevated temperature in oils that have a melting point above body temperature (preferably above 50° C.), for example compritol, and can be readily dispersed in these oils in a second step.

After dispersing the particles in oil, an emulsion with water as continuous phase can be prepared in a third step with suitable emulsifiers such as lipids, blockpolymers or poloxamers, or other polymers. The crude emulsion can be processed first in an ultra turrax, or ultrasound before processing it through an high pressure homogenizer. Examples of the latter are e.g. the Microfluidizer system or the APV Gaulin system. In case an oil phase is used that melts at elevated temperature, the whole process has to be performed at high temperature. After processing, an emulsion is obtained with stabilized oil droplets each containing multiple magnetic particles. Typical sizes of these oil droplets are between 80 to 500 nm, preferably between 100 to 200 nm in diameter. By controlling the amount of dispersed magnetic particles in the oil phase, the number of particles inside an individual emulsion droplet can be controlled to a good (averaged) degree. For example, an emulsion droplet of about 200 nm in diameter can contain up to 1000 particles, each with a diameter of about 20 nm, or less if desired.

In case of an emulsion with an oil that is liquid at bodytemperature, the magnetic particles will be dispersed inside randomly. In case of an emulsion droplet, that is based on oil which melts at higher temperature and solidifies at body temperature, the magnetic particles are initially also statistically dispersed in the solidified oil phase for temperatures below the melting temperature. The latter type is referred to as solid emulsion particles.

Above systems form the basis of new tracer materials for MPI as the interaction of a controlled number of magnetic particles, each with intrinsic special magnetic properties, will lead to new magnetic properties of the whole entity.

The tracer materials based on solid emulsion particles, can be further manipulated in a magnetic field to even further tune their magnetic response in MPI. For example, solid emulsions particles which are suspended in a water-based medium, or any other suitable continuous phase, can be brought into a magnetic field (AC or DC) and heated above the melting temperature of the oil. Upon melting, the magnetic particles can for example align in the magnetic field or agglomerate or cluster or interact in a special way, or partially align in case an AC field is used. In the latter case, only the good responsive particles align. The system may then be quenched below the melting temperature of the oil to “freeze” the magnetic particles in the solidifying oil. The performance of such systems changes significantly when anisotropic magnetic materials are used.

The above-described principle can be extended to particles that are stabilized by a thin polymer shell, for example polylactic acid. The inner phase of these particles can be based on similar oils with dispersed magnetic particles as described above, with all further processing steps as described above. However, in an additional step, the oil can be removed by freeze drying, leaving behind clustered magnetic materials in the polymer shell. These types of entities will show new properties in MPI.

A magnetic tracer material may also be based on liposomes or polymersomes, or vesicle based systems: liposome or polymersome, or vesicle based systems are formed by amphiphilic molecules and self assemble into vesicles with an inner volume of water separated by a hydrophobic membrane from the outside. Magnetic particles that are coated with a hydrophobic coating, as described with reference to the first step above, can be incorporated into the hydrophobic membrane and can thus be arranged basically on the surface of a sphere. Typical sizes of the vesicles can be from 60 to 500 nm with a corresponding increase of magnetic particles. A typical liposome solution contains about 2% lipids by weight. A typical example is 60% mol phosphatidylcholin, 30% mol cholesterol and 10% mol phosphatidylethanolamine or natural products like egg yolk phospholipids.

The lipids may be dissolved in CH₂Cl₂ and a certain amount of magnetic particles dispersed in CH₂Cl₂ be added. The mixture is dried e.g. at a RotorVap in a round bottom flask in order to form a film on the glass, and subsequently dried under vacuum. The film is rehydrated with a water based solution, e.g. water containing a buffer of stabilizers, and crude mixed in an ultra turrax. Subsequently the mixture is processed in an extruder under high pressure or in a high pressure homogenizer to form a liposome system. Due to self assembly, the magnetic particles are incorporated in the hydrophobic membrane. The 2D arrangement of these particles will lead to new magnetic properties that lead to a different behavior in MPI. The lipid membrane has a thickness of ca. 4 nm and allows to incorporate only rather small magnetic particles with sizes around 2-4 nm. If bigger magnetic particles need to be incorporates, polymersomes offer advantages as they can be prepared with thicker hydrophobic membranes.

Polymersomes can be prepared using e.g. amphiphilic polymers. A well studied example is the polymer diblock system polyethyleneoxide-bolybutadiene. The hydrophobic molecular weight fraction f_{philic}, i.e. the molecular weight of the hydrophobic part divided by the total molecular weight, needs to be in the range of ca. 0.2<f_{philic}<0.4, in order to form vesicular morphologies. For higher values of f_{philic}, other structures like cylindrical micelles or micelles are formed that offer again different properties. The molecular weight of the polyethylene oxide (PEO) part is preferably in the range 500<Mw, PEO<5000. Larger Mw are possible but may show less preferred properties in biodistribution and organ retention times for in-vivo applications. The preparation of polymersomes follows recipe outlined above for liposomes. Before extruding the polymersomes, it is advantageous to include a freeze-thaw cycle, by placing the crude polymer-water dispersion into liquid nitrogen bath and subsequently in a water bath at 60 degree. The freeze thaw cycle should be repeated around 5 times to yield smaller vesicles that can subsequently be extruded

Alternatively, magnetic particles that are hydrophilic, e.g Resovist, can be incorporated in the inner water-compartment of vesicles, liposomes or polymersomes. To do so, the particles having a hydrophilic coating are added to the water when hydrating the lipid film in the above-described production sequence. After processing, magnetic particles are arranged in the inner compartment of the liposomes or polymersomes. The remaining magnetic particles in the outer solution can be removed by processing the mixture over a column to remove the non-incorporated magnetic particles. Such as system will have new properties in MPI.

While the invention has been illustrated and described in detail in the drawings and foregoing description, such illustration and description are to be considered illustrative or exemplary and not restrictive; the invention is not limited to the disclosed embodiments. Variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage.

1. A magnetic tracer material for use in magnetic particle imaging comprising a plurality of magnetic particles that are clustered in a controlled way to form individual entities.

2. The material according to claim 1, wherein the magnetic particles comprise a magnetic material, such as Fe, Co, Ni, a composition, such as FeₓO_{y}, e.g. Fe₂O₃ or Fe₃O₄, or doped materials, such as Co, Ni, Zn, or Mn:FeₓO_{y}, or ferrite materials.

3. The material according to claim 1, wherein the magnetic particles have an average size of about 20-50 nm.

4. The material according to claim 1, wherein the magnetic particles have an average size of about 5-10 nm which in turn interact to form particles having an equivalent size of about 20-50 nm.

5. The material according to claim 1, wherein the individual entities comprise stabilized oil droplets, solid emulsion particles, liposomes, polymersomes or vesicles.

6. The material according to claim 1, where clustering is inside a naturally occurring biological entity such as a cell, red blood cell, virus etc.

7. The material according to claim 1, wherein the individual entities have an average size between 10 and 1000 nm, preferably between 100 to 200 nm in diameter.

8. A method for manufacturing a magnetic tracer material for use in magnetic particle imaging, comprising the steps of
dispersing magnetic particles in a spatially delimited medium and
clustering a plurality of the magnetic particles in a controlled way to form individual entities.

9. The method according to claim 8, wherein the magnetic particles comprise a magnetic material, such as Fe, Co, Ni, a composition such as FeₓO_{y}, e.g. Fe₂O₃ or Fe₃O₄, or doped materials, such as Co, Ni, Zn, or Mn:FeₓO_{y}.

10. The method according to claim 8, wherein the magnetic particles are coated with a hydrophobic surface coating.

11. The method according to claim 8, wherein the magnetic particles are dispersed in an organic solvent, preferably a hydrophobic organic solvent, such as toluene, heptane or CH₂Cl₂.

12. The method according to claim 11, wherein a controlled amount of magnetic particles is further dispersed in a hydrophobic oil, such as safflower oil or poppy seed oil, or (partially) fluorinated oils.

13. The method according to claim 12, further comprising preparing an emulsion of the magnetic particles dispersed in hydrophobic oil with water, preferably using an emulsifier such as a lipid, a blockpolymer, a poloxamer or another suitable polymer, so that the individual entities of clustered magnetic particles are formed in the emulsion in the form of stabilized oil droplets including a controlled amount of magnetic particles.

14. The method according to claim 13, wherein an oil having a melting point above body temperature, preferably above 50° C., is used, so that the stabilized oil droplets form solid emulsion particles at temperatures below the melting point of the oil.

15. The method according to claim 14, wherein the solid emulsion particles are further manipulated by heating the solid emulsion particles suspended in a medium above the melting point of the oil, subjecting the suspension to an external magnetic field and again cooling the suspension to conserve the magnetic properties.

16. The method according to claim 14, further comprising the step of removing the oil, preferably by freeze drying the emulsion, so that clustered magnetic particles stabilized in a polymer shell are formed.

17. The method according to claim 10, wherein the magnetic particles are dispersed in CH₂Cl₂ and mixed with a solution comprising lipids or amphiphilic polymers, said mixture being further processed to form the individual entities of clustered magnetic particles in the form of liposomes, polymersomes or vesicles having an inner volume of water separated by a hydrophobic membrane, wherein the magnetic particles are controlled to be arranged in the hydrophobic membrane.

18. The method according to claim 8, wherein the, preferably hydrophilic, magnetic particles are mixed with a solution comprising lipids or amphiphilic polymers, said mixture being further processed to form the individual entities of clustered magnetic particles in the form of liposomes, polymersomes or vesicles having an inner volume of water separated by a hydrophobic membrane, wherein the magnetic particles are controlled to be arranged in the inner volume.
